(19) United States
(12) Patent Application Publication
(10) Pub. No.: US 2002/0133009 A1
(43) Pub. Date: Sep. 19, 2002

(54) Method for opening potassium channels

(76) Inventors: George N. Lambrou, Strasbourg (FR); Anna Ottlecz, Munchenstein (CH); Christine Percicot, Strasbourg (FR); Michael Wiederholt, Berlin (DE)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/090,085

(22) Filed: Mar. 4, 2002

Related U.S. Application Data
(60) Provisional application No. 60/275,316, filed on Mar. 13, 2001.

(51) Int. Cl. A61K 31/497; A61K 31/4965; A61K 31/506; A61K 31/443; A61K 31/357; A61K 31/5575

(52) U.S. Cl. 544/405; 514/255.05; 514/463; 514/256; 514/336; 544/333; 546/283.7; 514/573; 514/690; 549/446; 562/504; 568/379

(57) ABSTRACT

The present invention relates to a method for opening potassium channels in mammalian cells by administering to a mammal effective amounts of potassium channel-opening keto compounds as described herein.

### FIELD OF THE INVENTION

The present invention relates to a method for opening potassium channels in mammalian cells.

### BACKGROUND OF THE INVENTION

Thirty or more different potassium channels exist in mammalian cells in a variety of biological tissues, e.g., in the cell membranes of neuronal cells, smooth muscle cells, and islets of Langerhans in the pancreas. These potassium channels are involved in the modulation of various physiological processes and play a complex and critical role in normal cellular ionic homeostasis. There are a number of different potassium channel subtypes. One of the most important physiologically is the high-conductance Ca2+-activated K+ (Maxi-K) channel which is present in neuronal tissue and smooth muscle. Maxi-K channels are opened by an increase in intracellular calcium ion concentration or by membrane depolarization. Elevation of the intracellular calcium ion concentration is required for neurotransmitter release and for smooth muscle contraction. Therefore, modulation of Maxi-K channels affects neurotransmitter release from nerve terminals and the contraction of various smooth muscle tissues. A Maxi-K channel opening compound is one that will hyperpolarize a cell membrane by allowing potassium out of a cell, thereby inhibiting neuronal firing, transmitter release, and smooth muscle cell contraction.

Several compounds that open potassium channels, including the Maxi-K channel, are known, such as carboxyimidamide derivatives, which are useful as hypotensive agents, coronary vasodilators, and for ameliorating ophthalmic circulatory disturbances (U.S. Pat. No. 5,166,347, WO98/29135, expressly incorporated by reference herein in their entirety). The potassium channel opening compounds such as pinacidil and cromakalim are also known to decrease intraocular pressure (WO89/10757).

The present inventors have now surprisingly discovered that the keto compounds of the invention, set forth below, also open Maxi-K channels.

### SUMMARY OF THE INVENTION

The present invention is directed to a method for opening potassium channels which comprises administering an effective amount of a potassium channel opening keto compound as disclosed herein. In particular, the invention is directed to a method for opening the mammalian Maxi-K channel by administering an effective amount of a keto compound as disclosed herein.

Furthermore, the present invention relates to a method of maintaining or inducing hyperpolarization of the cell membrane which comprises administering an effective amount of a potassium channel opening keto compound as disclosed herein.

The present invention further relates to a method for treating conditions and disease states related to potassium channel function which comprises administering an effective amount of a potassium channel opening keto compound disclosed herein.

The present invention also relates to a potassium channel opening composition which comprises a keto compound disclosed herein as an active ingredient.

The present invention further relates to a use of a keto compound disclosed herein for opening potassium channels.

### DETAILED DESCRIPTION OF THE INVENTION

All patents, patent applications, and other publications referred to in this specification are expressly incorporated by reference in their entirety. In the event of a conflict between the present specification.

Terms are used in the present specification as follows.

The term “unsaturated” in the definitions for variables R1 and Ra in the structural formulae below is intended to include at least one or more double bonds and/or triple bonds that are isolatedly, separately or serially present between carbon atoms of the main and/or side chains.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms for R1 and 1 to 10, especially 1 to 8 carbon atoms for variable Ra.

The term “halogen atom” covers fluorine, chlorine, bromine and iodine. Particularly preferable is a fluorine atom.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O-, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-l-hydroxyethyl.

The term “lower alkanoyloxy” refers to a group represented by the formula RCO-O-15, wherein RCO- is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O-, wherein cyclo(lower)alkyl is as defined above.

The term “aryl” may include unsubstituted or substituted aromatic hydrocarbon rings (preferably monocyclic groups), for example, phenyl, naphthyl, tolyl, xylyl. Examples of the substituents are halogen atom and halo(lower)alkyl, wherein halogen atom and lower alkyl are as defined above.

The term “aryloxy” refers to a group represented by the formula ArO-, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazyl, pyrimidyl, pyrazyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, puryl, quinazolinyl, carbazolyl, acridinyl, phenanthridinyl, benzimidazolyl, benzimidazolonyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO-, wherein Hc is a heterocyclic group as described above.

The term “functional derivative” includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and I-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tosyl ester, t-butylphenyl ester, salicyl ester, 3,4-di-methoxyphenyl ester and benzamidophenyl ester; and aryl(lower)alkyl ester such as benzyl ester, trityl ester and benzhydryl ester. Examples of the amides are mono- or di-lower alkyl amides such as methylamide, ethylamide and dimethylamide; arylamides such as anilide and toluidide; and alkyl- or aryl-sulfonylamides such as methylsulfonylamide, ethylsulfonyl-amide and tolylsulfonylamide.

Preferred potassium channel opening keto compounds that can be utilized in the practice of the present invention are represented by the formula:

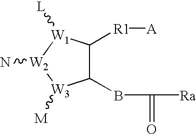

wherein W1, W2 and W3 are carbon or oxygen atoms,

L, M and N are a hydrogen atom, hydroxy, halogen atom, lower alkyl, lower alkoxy, hydroxy(lower)alkyl, or oxo, wherein at least one of L and M is a group other than hydrogen, and the five-membered ring may have at least one double bond;

A is —CH₂OH, —COCH₂OH, —COOH or a functional derivative thereof;

B is single bond, —CH₂—, —CH₂—CH₂—, —CH═CH—, —C≡C—, —CH₂—CH₂—CH₂—, —CH═CH—CH₂—, —CH₂—CH═CH—, —C≡C—CH₂—, or —CH₂—C≡C—;

R1 is a saturated or unsaturated bivalent lower or medium aliphatic hydrocarbon residue, which is unsubstituted or substituted with halogen, an alkyl group, hydroxy, oxo, aryl or heterocyclic group ; and

Ra is cyclo(lower)alkyl, cyclo(lower)alkyloxy, aryl, aryloxy, a heterocyclic group, a heterocyclic-oxy group, a saturated or unsaturated lower or medium aliphatic hydrocarbon residue which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, a heterocyclic group or a heterocyclic-oxy group.

A group of particularly preferable compounds among the above described compounds is represented by the formula:

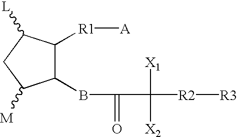

wherein L, M, R1, A and B are the same definition described above,X1 and X2 are hydrogen, lower alkyl, or halogen;R2 is a single bond or lower alkylene; and

R3 is lower alkyl, lower alkoxy, cyclo(lower)alkyl, cyclo(lower)alkyloxy, aryl, aryloxy, heterocyclic group or heterocyclic-oxy group.

Preferred values for L and M include hydroxy and oxo. L and M are more preferably both hydroxy.

Preferred values for A are —COOH, —CH2OH, or a salt, ester, ether or amide thereof.

It is preferred with respect to variables X1 and X2 that at least one be halogen, and it is more preferred that both X1 and X2 are halogen. It is most preferred that both X1 and X2 are fluorine.

It is preferred that variable RI be an unsubstituted saturated or unsaturated bivalent lower-medium aliphatic hydrocarbon residue. More preferably, RI has 1-10 carbon atoms, most preferably 2-8 carbon atoms.

Examples of RI include the following groups:

—CH₂—CH₂—,

—CH₂—CH₂—CH₂—CH₂—,

—CH₂—CH═CH—CH₂—,

—CH₂—C≡C—CH₂—,

—CH₂—CH₂—CH₂—CH₂—CH₂—,

—CH₂—CH═CH—CH₂—CH₂—,

—CH₂—C≡C—CH₂—CH—,

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

CH₂—C≡C—CH₂—CH₂—CH₂—,

CH₂—CH₂—CH₂—CH₂—CH(CH3)—CH ₂—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—,

—CH₂—CH₂—CH₂—CH₂—CH₂—CH₂—CH₂(CH3)—CH₂

It is preferred that variable R2 be a single bond or a saturated or unsaturated bivalent lower-medium aliphatic hydrocarbon residue. If variable R2 is a saturated or unsaturated bivalent lower-medium aliphatic hydrocarbon residue, then variable R2 preferably has 1-10 carbon atoms, more preferably 1-8 carbon atoms.

Preferred values for variable R3 are hydrogen, aryl, or aryloxy.

It is preferred that variable Ra is a hydrocarbon containing 1-10 carbon atoms, more preferably, a hydrocarbon containing 1-8 carbon atoms and, most preferably, a hydrocarbon containing 7 carbon atoms.

Other preferred values for Ra is straight chain hydrocarbon with at least 6 carbon atoms, or straight chain carbon with at least 3 carbon atoms terminating with a carbon ring, more preferably a phenyl ring.

A particularly preferred compound is unoprostone isopropyl, [1R-[1α(Z),2β,3a,5a]]-7-[3,5-dihydroxy-2-(3-oxodecyl)cyclopentyl]-5-heptenoic acid isopropyl ester, with the formula

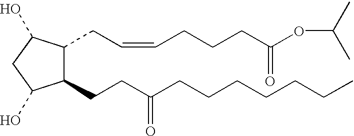

In the present invention, any isomers such as the individual tautomeric isomers, the mixture thereof, or optical isomers, the mixture thereof, a racemic mixture, and other steric isomers may be used for the same purpose.

The compounds used in the present invention may be prepared utilizing the methods disclosed in U.S. Pat. Nos. 5,073,569, 5,166,174, 5,221,763, 5,212,324 and 5,739,161 and U.S. patent application Ser. No. 09/011,218 in combination with the knowledge of those of ordinary skill in the art.

The term “treatment” used herein refers to any means of control of a condition including prevention, cure, relief of the condition, and prevention or relief of development or progression of the condition.

The potassium channel opening keto compounds, and compositions comprising such compounds, disclosed herein may be used for treatment of disorders involving potassium channels in humans and other mammals. Usually, it is administered systemically or topically by oral administration, intravenous injection (including infusion), subcutaneous injection, intra rectal administration, intra vaginal administration, ophthalmic administration (including ophthalmic ointment) and the like. Considering the systemic affection and effectiveness of the treatment, a dosage form for ophthalmic administration is especially preferable.

The dosage form of the composition may be eye drops, ophthalmic ointment, powders, granules, tablets, capsules, suppository, vaginal suppository, injection and ointment, and especially eyedrops and ophthalmic ointment are preferable. These dosages forms may be prepared according to any of conventional methods.

According to the present invention, the “effective amount” of the composition means the amount necessary for desired treatment and may vary depending on the species of the subject, such as human or animals, to be treated, age, body weight, condition to be treated, desired effect, administration technique, period for treatment and the like. A satisfactory effects may be obtained by topical administration of the compound at the amount of 0.0001-1000 μg/eye, or by systemic administration 2-4 times per day or continuous administration at the amount of 0.00001-100 mg/Kg per day.

The compositions of the present invention may contain a potassium channel opening keto compound as sole active ingredient, or may contain one or more additional pharmaceutically active ingredients. The amount of the respective ingredients may be suitably controlled based on their therapeutic effects and safety.

The composition of the present invention may further contain physiologically acceptable additives. Said additives may include excipient, diluent, filler, resolvent, lubricant, adjuvant, binder, disintegrator, coating agent, capsulating agent, ointment base, suppository base, aerozoling agent, emulsifier, dispersing agent, suspending agent, tonicity agent, buffering agent, soothing agent, preservative, antioxidant, corrigent, flavor, colorant, a functional material such as cyclodextrin and biodegradable polymer, stabilizer, pH modifier and chelating agent. The additives may be selected from those described in general reference books of pharmaceutics.

The potassium channel opening keto compounds, and compositions comprising such compounds, of the present invention are applied to the treatment of the conditions and disease states related to the function of potassium channels and depolarization of cell membranes, such as hypertension, pulmonary hypertension, asthma, interstitial cystitis, urinary incontinence and other urogenital disorders, ischemic bowel disease, gastrointestinal motility disorders, arrhythmias, peripheral vascular disease, congestive. heart failure, dysmenorrhea, optic nerve disorder, glaucoma, ocular hypertension, angina, and alopecia.

The present invention will be illustrated in more detail by way of the following examples. These examples should not be used as any limitation of the present invention.

### EXAMPLE

Maxi K Channel Opening Activity of Unoprostone Isopropyl

Patch clamp experiments of cultured human and bovine trabecular meshwork (TM) cells are performed. Nystatin in a pipette is used to obtain perforated patches. Whole-cell current measurements in the cells are performed. Voltages from −80mV to +120mV with 200-msec duration each are applied through the patch pipette, starting from a holding potential of −40mV. After each step, the voltage is returned to a holding potential of 40 mV for 200-msec. Currents are continuously sampled at 100 Hz throughout the duration of the protocol.

In bovine and human trabecular meshwork (TM) cells, unoprostone isopropyl stimulated the overall current of the cells incubated in either acetylcholine or endothelin.

The highly specific inhibitor of maxi-K+channels, iberiotoxin, totally reduced the unoprostone isopropyl -induced current. The data show that unoprostone isopropyl directly interacts with the maxi-K+ channels and that unoprostone isopropyl opens maxi-K+ channels.

In bovine trabecular meshwork cells incubated in acetylcholine, the total current is significantly stimulated to approximately 180% (** p<0.01) of the control by 10-5 M unoprostone isopropyl. The total current is inhibited by iberiotoxin to approximately 40% of the control. After washout of iberiotoxin the current recovers to the original current after several minutes.

When human trabecular meshwork cells are incubated in acetylcholine, unoprostone isopropyl stimulates the overall current which can be inhibited by the highly specific inhibitor of K+ channels, iberiotoxin.

When human trabecular meshwork cells are incubated in the presence of endothelin and unoprostone isopropyl, unoprostone isopropyl induces an increase in the overall current which can be inhibited by iberiotoxin.

What is claimed is:

1. A method for opening potassium channels in the cell membranes of a mammal in need of such treatment comprising administering to the mammal an effective amount of a compound with the formula: 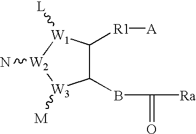
wherein W1, W2 and W3 are carbon or oxygen atoms,
L, M and N are a hydrogen atom, hydroxy, halogen atom, lower alkyl, lower alkoxy, hydroxy(lower)alkyl, or oxo, wherein at least one of L and M is a group other than hydrogen, and the five-membered ring may have at least one double bond;
A is —CH₂OH, —COCH₂OH, —COOH or a functional derivative thereof;
B is single bond, —CH₂—, —CH₂—CH₂—, —CH═CH—, —C≡C—, —CH₂—CH₂—CH₂—, —CH═CH—CH₂—, —CH₂—CH═CH—, —C≡C-CH₂—, or —CH₂—C≡C—;
R1 is a saturated or unsaturated bivalent lower or medium aliphatic hydrocarbon residue, which is unsubstituted or substituted with halogen, an alkyl group, hydroxy, oxo, aryl or heterocyclic group ; and
Ra is cyclo(lower)alkyl, cyclo(lower)alkyloxy, aryl, aryloxy, a heterocyclic group, a heterocyclic-oxy group, or a saturated or unsaturated lower or medium aliphatic hydrocarbon residue which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, a heterocyclic group, or a heterocyclic-oxy group.

2. A method for opening potassium channels in the cell membranes of a mammal in need of such treatment comprising administering to the mammal an effective amount of a compound with the formula: 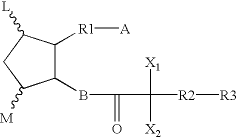
wherein L, and M are a hydrogen atom, hydroxy, halogen atom, lower alkyl, lower alkoxy, hydroxy(lower)alkyl, or oxo, wherein at least one of L and M is a group other than hydrogen, and the five-membered ring may have at least one double bond;
A is —CH₂OH, —COCH₂OH, —COOH or a functional derivative thereof;
B is single bond, —CH₂—, —CH₂—CH₂—, —CH═CH—, —C≡C—, —CH₂—CH₂—CH₂—, —CH═CH—CH₂—, —CH₂—CH═CH—, —C≡C—CH₂—, or —CH2—C≡C—;
R1 is a saturated or unsaturated bivalent lower or medium aliphatic hydrocarbon residue, which is unsubstituted or substituted with halogen, an alkyl group, hydroxy, oxo, aryl or a heterocyclic group; and
X1 and X2 are hydrogen, lower alkyl, or halogen;
R2 is a single bond or lower alkylene; and
R3 is lower alkyl, lower alkoxy, cyclo(lower)alkyl, cyclo(lower)alkyloxy, aryl, aryloxy, a heterocyclic group or a heterocyclic-oxy group.

3. A method for maintaining or inducing hyperpolarization in the cell membranes of a mammal in need of such treatment which comprises administering to the mammal an effective amount of a compound with the formula: 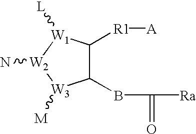
wherein W1, W2 and W3 are carbon or oxygen atoms,
L, M and N are a hydrogen atom, hydroxy, halogen atom, lower alkyl lower alkoxy, hydroxy(lower)alkyl, or oxo, wherein at least one of L and M is a group other than hydrogen, and the five-membered ring may have at least one double bond;
A is —CH₂₀H, —COCH₂₀H, —COOH or a functional derivative thereof;
B is single bond, —CH₂—, —CH₂—CH₂—, —CH═CH—, —C≡C—, —CH₂—CH₂—CH₂—, —CH═CH—CH₂—, —CH₂—CH═CH—, —C≡C—CH₂—, or —CH₂—C≡C—;
R1 is a saturated or unsaturated bivalent lower or medium aliphatic hydrocarbon residue, which is unsubstituted or substituted with halogen, an alkyl group, hydroxy, oxo, aryl or heterocyclic group ; and
Ra is cyclo(lower)alkyl, cyclo(lower)alkyloxy, aryl, aryloxy, a heterocyclic group, a heterocyclic-oxy group, or a saturated or unsaturated lower or medium aliphatic hydrocarbon residue which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, a heterocyclic group, or a heterocyclic-oxy group.

4. A method for treating conditions and disease states characterized by excessive cell membrane depolarization which comprises administering to the mammal an effective amount of a compound with the formula: 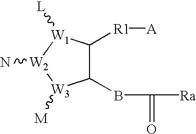
wherein W1, W2 and W3 are carbon or oxygen atoms,
L, M and N are a hydrogen atom, hydroxy, halogen atom, lower alkyl, lower alkoxy, hydroxy(lower)alkyl, or oxo, wherein at least one of L and M is a group other than hydrogen, and the five-membered ring may have at least one double bond;
A is —CH₂OH, —COCH₂OH, —COOH or a functional derivative thereof;
B is single bond, —CH₂—, —CH₂—CH₂—, —CH═CH—, —C≡C—, —CH₂—CH₂—CH₂—, —CH═CH—CH₂—, —CH₂—CH═CH—, —C≡C—CH₂—, or —CH₂—C≡C—;
R1 is a saturated or unsaturated bivalent lower or medium aliphatic hydrocarbon residue, which is unsubstituted or substituted with halogen, an alkyl group, hydroxy, oxo, aryl or heterocyclic group ; and
Ra is cyclo(lower)alkyl, cyclo(lower)alkyloxy, aryl, aryloxy, a heterocyclic group, a heterocyclic-oxy group, or a saturated or unsaturated lower or medium aliphatic hydrocarbon residue which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, a heterocyclic group, or a heterocyclic-oxy group.

5. The method of claim 1, wherein said compound is unoprostone isopropyl.

6. The method of claim 2, wherein said compound is unoprostone isopropyl.

7. The method of claim 3, wherein said compound is unoprostone isopropyl.

8. The method of claim 4, wherein said compound is unoprostone isopropyl.

9. The method of claim 1, wherein said condition or disease state is hypertension, pulmonary hypertension, asthma, interstitial cystitis, urinary incontinence and other urogenital disorders, ischemic bowel disease, gastrointestinal motility disorders, arrhythmias, peripheral vascular disease, congestive heart failure, dysmenorrhea, angina, or alopecia.

10. The method of claim 2, wherein said condition or disease state is hypertension, pulmonary hypertension, asthma, interstitial cystitis, urinary incontinence and other urogenital disorders, ischemic bowel disease, gastrointestinal motility disorders, arrhythmias, peripheral vascular disease, congestive heart failure, dysmenorrhea, angina, or alopecia.

11. The method of claim 3, wherein said condition or disease state is hypertension, pulmonary hypertension, asthma, interstitial cystitis, urinary incontinence and other urogenital disorders, ischemic bowel disease, gastrointestinal motility disorders, arrhythmias, peripheral vascular disease, congestive heart failure, dysmenorrhea, angina, or alopecia.

12. The method of claim 4, wherein said condition or disease state is hypertension, pulmonary hypertension, asthma, interstitial cystitis, urinary incontinence and other urogenital disorders, ischemic bowel disease, gastrointestinal motility disorders, arrhythmias, peripheral vascular disease, congestive heart failure, dysmenorrhea, angina, or alopecia.
